(19) United States
(12) Patent Application Publication
(10) Pub. No.: US 2024/0128895 A1
(43) Pub. Date: Apr. 18, 2024

(54) TRIBOELECTRIC NANOGENERATOR AND PREPARATION METHOD, SELF-POWERED SENSING SYSTEM, AND JOINT ANGLE DETECTION METHOD

(71) Applicant: SHENZHEN UNIVERSITY, Guangdong (CN)

(72) Inventors: Longbiao HUANG, Guangdong (CN); Jiancheng HAN, Guangdong (CN)

(73) Assignee: SHENZHEN UNIVERSITY, Guangdong (CN)

(21) Appl. No.: 18/271,859

(22) PCT Filed: Feb. 3, 2021
(86) PCT No.: PCT/CN2021/075024

§ 371 Date: Dec. 20, 2023

(30) Foreign Application Priority Data
Jan. 12, 2021 (CN) 202110038924.0

(51) Int. Cl. H02N 1/04; B33Y 10/00; B33Y 80/00

(52) U.S. Cl. CPC H02N 1/04; B33Y 10/00; B33Y 80/00; G01B 7/30

(57) ABSTRACT

Provided is a triboelectric nanogenerator based on a 4D printing technology, including a first substrate layer and first triboelectric components of friction units, the plurality of friction units being arranged at an interval by taking a geometric center of the first substrate layer as a center of a circle; and a second substrate layer and second triboelectric components of first electrodes and second electrodes, the plurality of first electrodes and second electrodes being arranged alternately by taking a geometric center of the second substrate layer as a center of a circle. There are gaps between the first electrodes and the second electrodes; and the first substrate layer and the second substrate layer are inserted into each other through respective flanges and grooves, so that the friction units are in contact friction with the first electrodes and the second electrodes.

### TECHNICAL FIELD

The present invention relates to the cross technical field of combining a 4D printing technology, a sensing technology, and a self-powered system, and particularly relates to a triboelectric nanogenerator based on a 4D printing technology, a self-powered sensing system, a method for detecting a rotation angle of a joint based on the self-powered system, and a method for preparing the triboelectric nanogenerator based on the 4D printing technology.

### BACKGROUND

With arrival of the era of the internet of things, a plenty of portable electronic devices are applied, and these electronic devices are usually powered by batteries. However, batteries need to be frequently charged or replaced for supplying power, and waste batteries also bring severe environmental pollution. Therefore, it is an urgent need of a self-powered sensing technology to realize detection without an external power supply. To solve the problem, a self-powered sensing system based on a triboelectric nanogenerator attracts widespread attention.

However, in the road to achieve universal use, there are still some problems for the self-powered sensing system based on a triboelectric nanogenerator. First, when an apparatus works for a long time, performance of the apparatus will attenuate, so that the detection result is mistaken. Second, the preparation process for the self-powered sensing system based on a triboelectric nanogenerator lags behind relatively. Finally, a conventional preparation process includes processing and assembling components to finally assemble an intact apparatus, but it is hard to guarantee the performance of apparatuses in a same batch by such a process, and finally, the detection effect is affected.

Patent literature CN111564985A (publication date: 21^{st} August 2020) discloses a sensing triboelectric nanogenerator, a sensing device for a tire and a force monitoring system, wherein the structure of the triboelectric nanogenerator is designed: substrate layers, electrode layers and friction layers are adhered in sequence to the inner wall of a flexible substrate layer to obtain a relatively close triboelectric nanogenerator; the friction layers are in contact with each other under the action of a pretightening force; when both sides of the substrate layers are subjected to a reverse acting force, the friction layers are away from each other to generate electric signals related to deformation quantity; and the deformation characteristic of the triboelectric nanogenerator can be judged according to the characteristics of the electric signals, so that the triboelectric nanogenerator disclosed by the invention has a sensing function. However, its preparation flow is complicated, the preparation efficiency and preparation precision are low, and the service life of the manufactured sensing device is unsatisfactory.

To promote application and development of the sensing device based on the triboelectric nanogenerator, there is an urgent need of a brand new process to improve the preparation efficiency and preparation precision of the sensing device and to prolong the service life of the sensing device.

### SUMMARY

The present invention is intended to at least solve the technical problems that a preparation flow of a sensing device is complicated and the preparation efficiency and precision are low, and the service life of the prepared sensing device is short to a certain extent.

The principal objective of the present invention is to provide a triboelectric nanogenerator based on a 4D printing technology. In order to solve the above technical problem, the present invention adopts the technical solution as follows:

A triboelectric nanogenerator based on a 4D printing technology, including a first triboelectric component and a second triboelectric component capable of rotating relative to each other, wherein the first triboelectric component includes a first substrate layer and friction units arranged on a surface of the first substrate layer, the plurality of friction units being arranged at an interval by taking a geometric center of the first substrate layer as a center of a circle; the second triboelectric component includes a second substrate layer, and first electrodes and second electrodes arranged on a surface of the second substrate layer, the plurality of first electrodes and second electrodes being arranged alternately by taking a geometric center of the second substrate layer as a center of a circle; and there are gaps between the first electrodes and the second electrodes; the first substrate layer and the second substrate layer are inserted into each other through respective flanges and grooves, so that the friction units are capable of rotating relative to the first electrodes and the second electrodes and being in contact friction with the first electrodes and the second electrodes; and the first triboelectric component and the second substrate layer are prepared by the 4D printing technology.

Preferably, a shape memory polymer or a self-repair material is used in the 4D printing technology for fused deposition printing, direct ink writing printing or digital light processing printing.

Preferably, there are bulges or grooves on a surface of each of friction units.

Preferably, a solution with a conducting substance is sprayed to the surface of the second substrate layer and the first electrodes and the second electrodes are obtained after volatilizing a solvent, and the conducting substance including a silver nanowire, a carbon nanotube or graphene.

Preferably, longitudinal cross-sections of the first triboelectric component and the second triboelectric component are polygonal or curved edge-shaped.

Preferably, a central angle corresponding to each of the friction units is a, and two adjacent friction units are spaced at a same central angle b; and a central angle corresponding to each of the first electrodes is c, and a central angle corresponding to each of the second electrodes is e, wherein a=c=d and b=c+2*e.

A further objective of the present invention is to provide a self-powered sensing system, wherein the triboelectric nanogenerator based on a 4D printing technology is assembled at a joint; the first triboelectric component and the second triboelectric component are assembled at one side of the joint; when the joint moves, the joint drives one of the triboelectric components and the other triboelectric component to rotate relative to each other, so as to generate an alternating current signal, and an angle of the joint movement can be deduced according to a characteristic of the alternating current signal.

A third objective of the present invention is to provide a method for detecting a rotation angle of a joint of the self-powered sensing system, including the following steps:
S1: measuring phases of electric signals outputted by the triboelectric nanogenerator at different rotation angles, respectively, and establishing a correspondence table between the “rotation angle” and the “phases of the outputted electric signals”;
S2: acquiring the outputted electric signals of the self-powered sensing system mounted at the joint in real time, and performing smoothing and noise reduction processing on the outputted electric signals;
S3: detecting phase information corresponding to the outputted electric signals subjected to smoothing and noise reduction processing in S2;
S4: matching the phase information of the outputted electric signals according to the correspondence table between the “rotation angle” and the “phases of the outputted electric signals” calibrated in S1; and
S5: acquiring the rotation angle of the joint according to a matching result between the phase information of the outputted electric signals and the rotation angles in S4.

Preferably, in S2, a plurality of self-powered sensing systems is mounted at the joint simultaneously, and correspondingly, a mean value of the phase information corresponding to the plurality of outputted electric signals subjected to smoothing and noise reduction processing is solved in S3 as final phase information.

A fourth objective of the present invention is to provide method for preparing a triboelectric nanogenerator based on a 4D printing technology, including the following steps:
S1: designing an independent layer-based triboelectric nanogenerator model;
S2: force analysis is performed on a working process of the model after modeling and a simulation test is performed on distribution of an electric potential field;
S3: importing the tested model into slicing software for slicing and layering, selecting a processing sequence according to an actual structure of the model and generating a processing instruction;
S4: importing the processing instruction into a 3D printer to complete print processing of the first triboelectric component and the second substrate layer, respectively; if a printed product in a processing process does not meet a use requirement, returning to S1 to complete design and simulation test of the model again and generating a new processing instruction;
S5: spraying a volatile solution doped with a conducting substance to the bottom surface of the second substrate layer by using a spraying machine after print processing, and volatilizing a solvent to obtain the first electrodes and the second electrodes; and
S6: assembling the first triboelectric component and the second triboelectric component with the first electrodes and the second electrodes into the triboelectric nanogenerator.

Compared with the prior art, the present invention has the following beneficial effects:
1. The 4D printing technology is introduced into preparation of the triboelectric nanogenerator, and an independent layer-based triboelectric nanogenerator is designed and prepared. First, by introducing the 4D printing technology, the triboelectric nanogenerator is prepared in a personalized manner with high precision and high efficiency, and surface bulges or grooves are formed on the surfaces of the friction units to increase the contact area, so that the output performance of the triboelectric nanogenerator is improved; second, the triboelectric nanogenerator prepared from a shape memory material further has a shape memory function; when there is performance attenuation due to deformation of the apparatus in a using process, the shape of the apparatus can be recovered by placing the deformed apparatus in a certain condition, so does the performance, thereby indirectly prolonging the service life of the triboelectric nanogenerator.
2. The independent layer-based triboelectric nanogenerator is assembled at the joint as the self-powered sensing system, and combined with a novel method for detecting the rotation angle of the joint, the triboelectric nano generator is used in a self-powered sensor for detecting joint movement. Different from a previous method for achieving detection of the sensing type triboelectric nanogenerator by detecting the intensity of the outputted signal, started from the principle of the outputted signal of the triboelectric nanogenerator, the angle of the joint movement is judged according to a relationship between the relative rotation angle between triboelectric assemblies and the characteristics of the outputted electric signals. Such a manner can effectively avoid detection errors caused by performance attention of the apparatus, so that the detection reliability is improved.

### BRIEF DESCRIPTION OF DRAWINGS

FIG. 1 is a top view of a surface of a first triboelectric component of a triboelectric nanogenerator based on a 4D printing technology provided in Embodiment 1 of the present invention.

FIG. 2 is a top view of a surface of a second triboelectric component of the triboelectric nanogenerator based on the 4D printing technology provided in Embodiment 1 of the present invention.

FIG. 3 is a schematic diagram of a cross section of the triboelectric nanogenerator based on the 4D printing technology provided in Embodiment 1 of the present invention.

FIG. 4 is a schematic diagram of a first step of a workflow of the triboelectric nanogenerator based on the 4D printing technology provided in Embodiment 1 of the present invention.

FIG. 5 is a schematic diagram of a second step of the workflow of the triboelectric nanogenerator based on the 4D printing technology provided in Embodiment 1 of the present invention.

FIG. 6 is a schematic diagram of a third step of the workflow of the triboelectric nanogenerator based on the 4D printing technology provided in Embodiment 1 of the present invention.

FIG. 7 is a schematic diagram of a fourth step of the workflow of the triboelectric nanogenerator based on the 4D printing technology provided in Embodiment 1 of the present invention.

FIG. 8 is an assembling schematic diagram of a self-powered sensing system provided in Embodiment 2 of the present invention for detecting a joint movement of a human body.

FIG. 9 is a variation diagram of a voltage of the self-powered sensing system provided in Embodiment 2 of the present invention.

FIG. 10 is a flowchart of steps of a method for detecting a rotation angle of a joint of the human body based on the self-powered sensing system provided in Embodiment 3 of the present invention.

FIG. 11 is a flow chart of steps of a method for preparing a triboelectric nanogenerator based on the 4D printing technology provided in Embodiment 4 of the present invention.

### DETAILED DESCRIPTION OF EMBODIMENTS

The drawings are merely used for exemplary description and are not construed as limitation to the patent.

In order to better describe the embodiments, some parts in the drawings will be omitted, amplified or lessened and the drawings do not represent the dimensions of actual products.

For those skilled in the art, it can be understood that some known structures and description thereof in the drawings may be omitted.

The technical solution of the present invention will be further described below in combination with the drawings and the embodiments.

### Embodiment 1

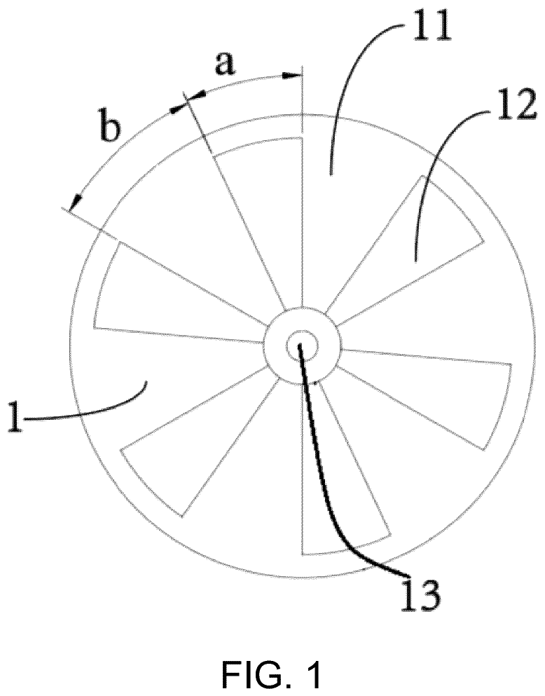

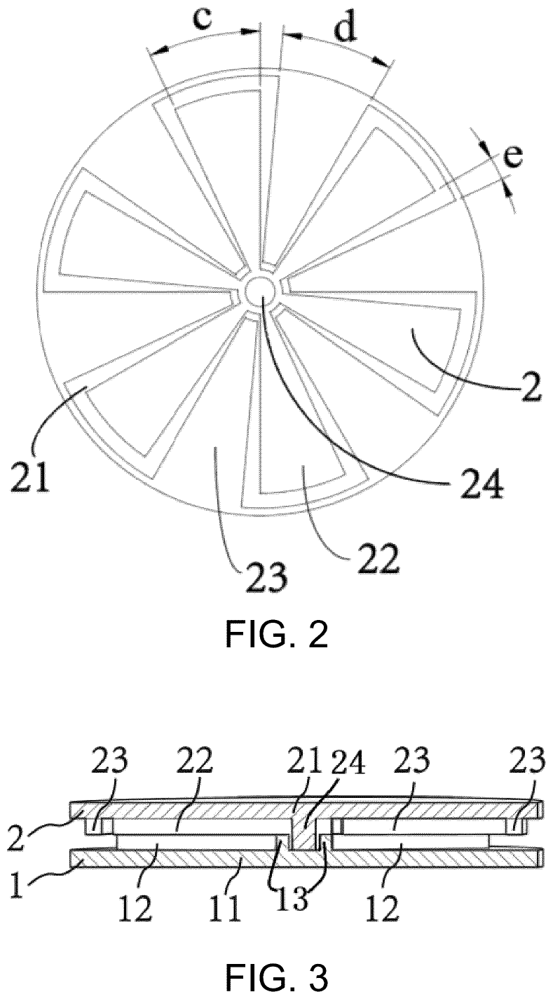

Referring to FIGS. 1-3, a triboelectric nanogenerator based on a 4D printing technology, including a first triboelectric component 1 and a second triboelectric component 2 capable of rotating relative to each other, wherein the first triboelectric component 1 includes a first substrate layer 11 and friction units 12 arranged on a surface of the first substrate layer 11, the plurality of friction units 12 being arranged at an interval by taking a geometric center of the first substrate layer 11 as a center of a circle; the second triboelectric component 2 includes a second substrate layer 21, and first electrodes 22 and second electrodes 23 arranged on a surface of the second substrate layer 21, the plurality of first electrodes 22 and second electrodes 23 being arranged alternately by taking a geometric center of the second substrate layer 21 as a center of a circle; and there are gaps between the first electrodes 22 and the second electrodes 23; the first substrate layer 11 and the second substrate layer 21 are inserted into each other through respective flanges 24 and grooves 13, so that the friction units 12 are capable of rotating relative to the first electrodes 22 and the second electrodes 23 and being in contact friction with the first electrodes and the second electrodes; and the first triboelectric component 1 and the second substrate layer 21 are prepared by the 4D printing technology.

Specifically, the first triboelectric component 1 and the second substrate 21 are made of a shape memory polymer or self-repair material. Further, a printing filament can be polyurethane. The shape memory polymers or the self-repair materials are printed by 3D printing methods such as fused deposition printing, ink direct writing printing or digital light processing printing. A solution containing a conducting substance is sprayed to the first electrodes 22 and the second electrodes 23 by using a spraying machine to obtain corresponding electrode patterns by means of a mask, and finally, corresponding electrode layers are obtained after volatilizing a solvent. The conducting substance includes a silver nanowire, a carbon nano tube or graphene. Further, specifically, the solution used for preparing the conducting layer can be a silver nanowire methanol solution.

Specifically, longitudinal cross-sections of the first substrate 11 and the second substrate 21 are polygonal or curved edge-shaped. Further, the longitudinal cross-sections can be arranged equilaterally polygonal or round. The friction unit 12 is rectangular, triangular or fan-shaped. A central angle corresponding to each of the friction units 12 is a, and two adjacent friction units 12 are spaced at a same central angle b; and a central angle corresponding to each of the first electrodes 22 is c, and a central angle corresponding to each of the second electrodes 23 is e, wherein a=c=d and b=c+2*e.

In a specific implementation process, the first triboelectric component 1 and the second triboelectric component 2 are assembled together through flanges 24 and grooves 13 formed at the geometric centers of the two, so that the friction units 12 are in contact with the first electrodes 22 and the second electrodes 23 to form an independent layer-based triboelectric nanogenerator. The flanges 24 and the grooves 13 have round-shaped sections. To increase the effective contact area, surface bulge or groove patterns can be obtained on the surfaces of the plurality of friction units 12 through the 4D printing technology, so that the output performance of the triboelectric nanogenerator is improved. When the first triboelectric component 1 and the second triboelectric component 2 rotate relative to each other, under the actions of triboelectrification and electrostatic induction, the triboelectric nanogenerator generates an alternating current.

The working principle of the 4D printing triboelectric nanogenerator in the embodiment will be described below.

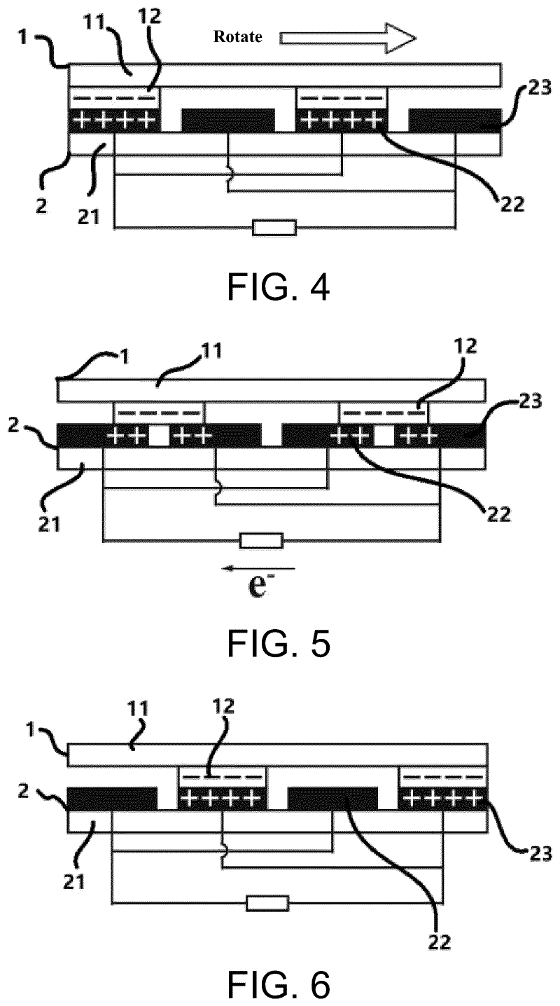

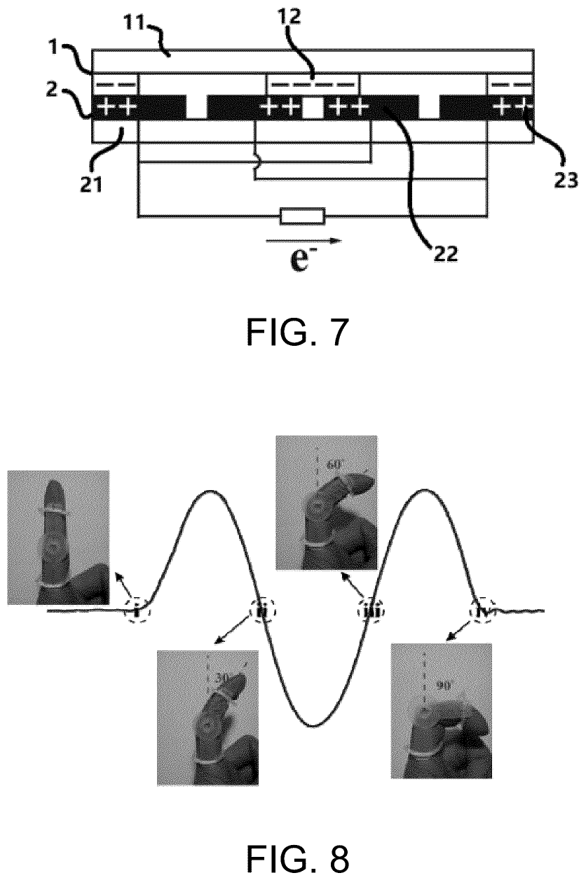

The working mode of the triboelectric nanogenerator in the embodiment is independent layer-based. As shown in FIGS. 4-7, under the action of an external force, the first electrodes 22 and the second electrodes 23 rotate relative to the friction units by taking the flanges and grooves as centers. In the rotating process, the plurality of friction units 12 are overlapped with the first electrodes 22 and the second electrodes 23 alternately to affect charge distribution on the surfaces of the electrodes, which induces a potential difference between the first electrodes 22 and the second electrodes 23. When the first electrodes 22 and the second electrodes 23 are electrically connected, the triboelectric nanogenerator generates an alternating current signal in an external circuit, and when the first electrodes 22 and the second electrodes 23 are open-circuited, the triboelectric nanogenerator outputs an alternating voltage signal outward. Specifically speaking, when friction occurs, the friction units have negative charges on the surfaces due to high electronegativity; when the plurality of friction units 12 are fully overlapped with the plurality of first electrodes 22, under the action of electrostatic induction, positive charges equivalent to those of the plurality of friction units 12 appear on the surfaces of the plurality of first electrodes 22, and in this case, there are no charges on the surfaces of the second electrodes 23, and therefore, there is a potential difference between the first electrodes 22 and the second electrodes 23. With continuous rotation of the friction units till the second electrodes 23 are fully covered, in this case, positive charges equivalent to those of the friction units 12 appear on the surfaces of the first electrodes 22, and there are no charges on the surfaces of the first electrodes 22, and therefore, there is a potential difference between the second electrodes 23 and the first electrodes 22; and when the external force takes effect continuously, the above power generation period will occur circularly.

Performance of the triboelectric nanogenerator is usually degraded due to deformation of a part of components, which greatly affects the working stability and the service life of the triboelectric nanogenerator. In the embodiment, by taking polyurethane as the printing filament, the triboelectric nanogenerator is prepared by the 4D printing technology. Thanks to the shape memory function, when the apparatus deforms to cause performance attenuation of the apparatus, after the deformed apparatus is heated at 60° C. for 1 min, the shape of the apparatus can be recovered. It is found by a test that the performance of the apparatus can be recovered as well.

### Embodiment 2

The embodiment provides a self-powered sensing system based on Embodiment 1, wherein the triboelectric nanogenerator based on a 4D printing technology is assembled at a joint of a human body; the first triboelectric component 1 and the second triboelectric component 2 are assembled at one side of the joint of the human body; when the joint of the human body moves, the joint drives one of the triboelectric components and the other triboelectric component to rotate relative to each other, so as to generate an alternating current signal, and an angle of the joint movement can be deduced according to a characteristic of the alternating current signal. Specifically, it is set that a=c=d=29°, b=31° and e=1°. In practical application, the detection precision of the self-powered sensing system will change with the above-mentioned parameters.

When the friction units 12 are located in the middle positions of the first electrodes 22 and the second electrodes 23, in this case, positive charges distributed on the surfaces of the first electrodes 22 are equivalent to positive charges distributed on the second electrodes 23. There is no potential difference between the two electrodes, in this case, corresponding to the point i in FIG. 8.

When the friction units 12 rotate at 15°, in this case, the friction units are fully overlapped with the first electrodes 22. Under the action of electrostatic induction, positive charges equivalent to negative charges on the surfaces of the friction units appear on the surfaces of the first electrodes 22, and in this case, the potential difference between the first electrodes 22 and the second electrodes 23 reaches a maximum value; when the friction units 12 rotate at 15° in a same direction, the friction units 12 arrive at the middle positions of the first electrodes 22 and the second electrodes 23 again, and in this case, there are equivalent positive charges on the surfaces of the first electrodes 22 and the second electrodes 23, respectively, and the potential difference between the electrodes disappear, corresponding to a potential difference variation curve between the point i to the point ii in FIG. 8.

When the friction units rotate at 30° in the same direction, the potential difference between the electrodes appears first and then returns to zero, corresponding to potential difference variation curves between the point ii to the point iii and between the point iii to the point iv in FIG. 8.

Therefore, by observing the rotation angles and the outputted waveforms of the friction units, we can find that when the relative rotation angles are respectively 30°, 60° and 90°, waveform phases generated by the sensor are respectively π, 2π and 3π. Therefore, we can know the relative rotation angles of the first triboelectric assembly and the second triboelectric assembly, i.e., the rotation angle of the joint, according to the phase characteristics of the outputted waveforms generated by the sensor.

It is worth noting that the relative rotation angle and the waveform phase are by no means in an invariable relationship (30°, π), (60°, 2π) and (90°, 3π). The specific corresponding relationship is affected by the specific structure of the apparatus, that is to say, the central angle corresponding to each of the friction units 12 is a, two adjacent friction units 12 are spaced at a same central angle b, a central angle corresponding to each of the first electrodes 22 is c, and a central angle corresponding to each of the second electrodes 23 is e, and these numerical values will affect the final judgment result.

In addition, the application scenarios of the self-powered sensing system are not limited to the joint of the human body but are various positions where rotation happens, for example, an arm of an industrial robot and the like. When being assembled at different joints, the triboelectric nanogenerator based on a 4D printing technology has different sizes. For example, the sizes of the triboelectric nanogenerators assembled at an elbow joint, a wrist joint or a finger joint change from big to small.

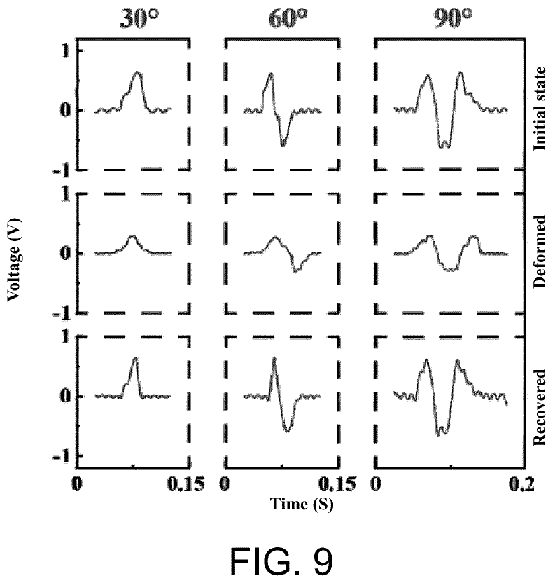

FIG. 9 shows a detection result of the self-powered sensing system at the finger joint. It can be seen that when the joint rotates at 30°, 60° and 90°, respectively, the waveform phases of the outputted electric signals of the sensor are n, 2n and 3π, respectively, and the peak voltage is maintained at about 0.7V; when the apparatus deforms, it can be seen that the phases of the outputted electric signals of the sensor do not change, and only the peak voltage decreases to about 0.3V, which is attenuated by about 55%; however, after the deformed sensor is heated at 60° C. for 1 min, the shape of the sensor is recovered. It can be seen from data in FIG. 8 that the performance of the sensor is recovered as well, the phase characteristics are same, and the peak voltage is recovered to about 0.7V.

### Embodiment 3

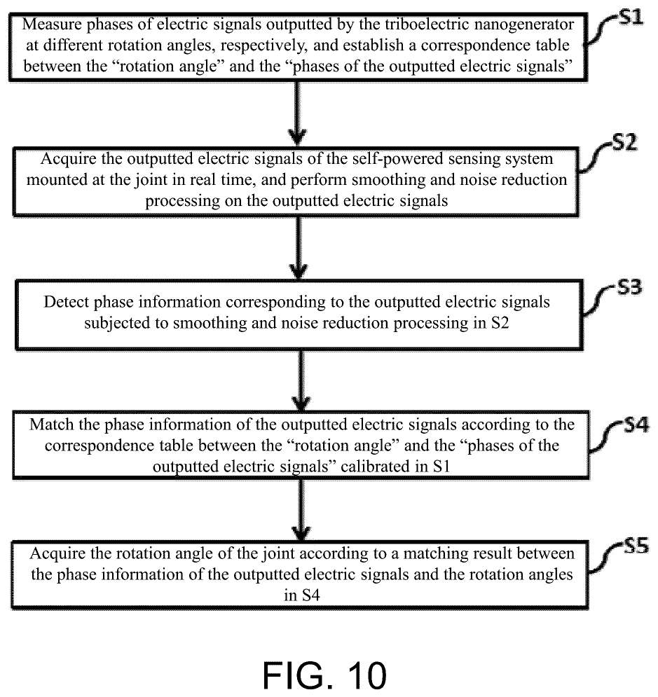

Referring to FIG. 10, the embodiment provides a method for detecting a rotation angle of a joint of a human body based on a self-powered sensing system, including the following steps:
S1: phases of electric signals outputted by the triboelectric nanogenerator at different rotation angles are measured, respectively, and a correspondence table between the “rotation angle” and the “phases of the outputted electric signals” is established;
S2: the outputted electric signals of the self-powered sensing system mounted at the joint are acquired in real time, and smoothing and noise reduction processing are performed on the outputted electric signals;
S3: phase information corresponding to the outputted electric signals subjected to smoothing and noise reduction processing in S2 are detected;
S4: the phase information of the outputted electric signals is matched according to the correspondence table between the “rotation angle” and the “phases of the outputted electric signals” calibrated in S1; and
S5: the rotation angle of the joint is acquired according to a matching result between the phase information of the outputted electric signals and the rotation angles in S4.

Specifically, in S2, a plurality of self-powered sensing systems can be mounted at the joint simultaneously, and correspondingly, a mean value of the phase information corresponding to the plurality of outputted electric signals subjected to smoothing and noise reduction processing is solved in S3 as final phase information.

The self-powered sensor provided by the embodiment adopts a novel detection method. Based on the working principle of the independent layer-based triboelectric nanogenerator, the moving condition of the joint is judged according to the phase relationship of the outputted waveforms of the triboelectric nanogenerator under different conditions. In the working process, even if performance is attenuated due to deformation of the apparatus, effective detection by the sensor is not affected, so that the reliability of sensor detection is improved.

### Embodiment 4

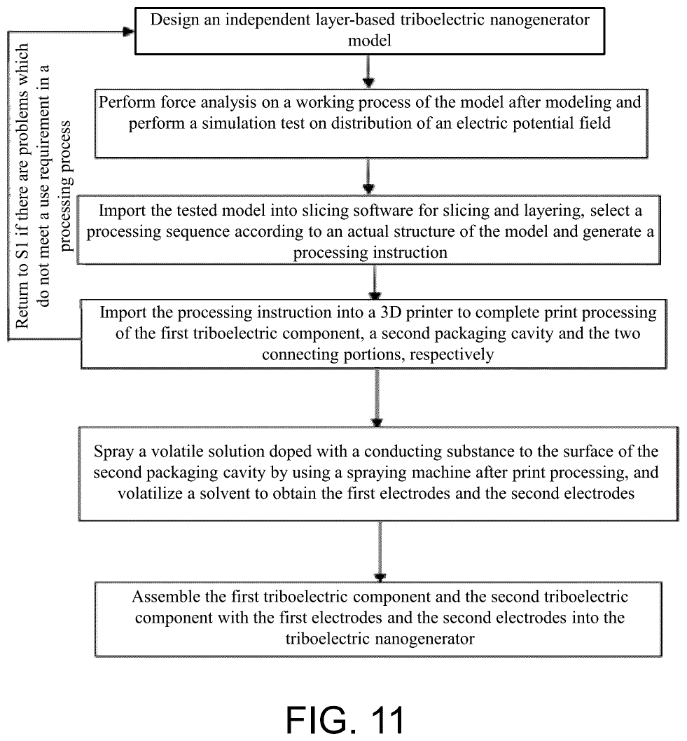

A method for preparing a triboelectric nanogenerator based on a 4D printing technology, referring to FIG. 11, including the following steps:
S1: an independent layer-based triboelectric nanogenerator model is designed;
S2: force analysis is performed on a working process of the model after modeling and a simulation test is performed on distribution of an electric potential field;
S3: the tested model is imported into slicing software for slicing and layering, a processing sequence is selected according to an actual structure of the model and a processing instruction (such as a gcode) is generated;
S4: the processing instruction is imported into a 3D printer to complete print processing of the first triboelectric component 1 and the second substrate layer 21, respectively; if a printed product in a processing process has the problems of collapse, deformation or influence on assembly, which does not meet a use requirement, it is returned to S1 to complete design and simulation test of the model again and generate a new processing instruction (such as the gcode);
S5: a volatile solution doped with a conducting substance is sprayed to the surface of the second substrate layer by using a spraying machine after print processing, and the first electrodes 22 and the second electrodes 23 are obtained after volatilizing a solvent; and
S6: the first triboelectric component 1 and the second triboelectric component 2 with the first electrodes 22 and the second electrodes 23 are assembled into the triboelectric nanogenerator.

Specifically, in S2, 3ds MAX software is used for modeling, and software such as COMSOL is used for performing force analysis on a working process of the model and performing simulation test on distribution of an electric potential field.

The above method for preparing a triboelectric nanogenerator based on a 4D printing technology can be also used for manufacturing triboelectric nanogenerators with various modes, including, but not limited to, a triboelectric nanogenerator with a transverse sliding mode, a triboelectric nanogenerator with a single electrode mode and a triboelectric nanogenerator with a contact-separating mode.

Same or similar marks correspond to same or similar parts.

The terms describing position relationships in the drawings are merely used for exemplary description and are not construed as limitation to the patent.

Apparently, the embodiments of the present invention are merely examples made for describing the present invention clearly and are not to limit the embodiments of the present invention. Those of ordinary skill in the art can further make modifications or variations in other forms on the basis of the above description. It is unnecessary to and unable to list all the implementation modes herein. Any modification, equivalent replacement, improvement, etc. made within the spirit and principle of the present invention shall be regarded as within the protection scope of the claims of the present invention.

1. A triboelectric nanogenerator based on a 4D printing technology, wherein the triboelectric nanogenerator comprises a first triboelectric component and a second triboelectric component capable of rotating relative to each other, wherein the first triboelectric component comprises a first substrate layer and friction units arranged on a surface of the first substrate layer, and the friction units are arranged at an interval by taking a geometric center of the first substrate layer as a center of a circle; the second triboelectric component comprises a second substrate layer, and first electrodes and second electrodes arranged on a surface of the second substrate layer, the first electrodes and second electrodes are arranged alternately by taking a geometric center of the second substrate layer as a center of a circle, and there are gaps between the first electrodes and the second electrodes; the first substrate layer and the second substrate layer are inserted into each other through respective flanges and grooves, so that the friction units are capable of rotating relative to the first electrodes and the second electrodes and being in contact friction with the first electrodes and the second electrodes; and the first triboelectric component and the second substrate layer are prepared by the 4D printing technology.

2. The triboelectric nanogenerator based on a 4D printing technology according to claim 1, wherein a shape memory polymer or a self-repair material is used in the 4D printing technology for fused deposition printing, direct ink writing printing or digital light processing printing.

3. The triboelectric nanogenerator based on a 4D printing technology according to claim 1, wherein there are bulges or grooves on a surface of each of the friction units.

4. The triboelectric nanogenerator based on a 4D printing technology according to claim 1, wherein a solution with a conducting substance is sprayed to the surface of the second substrate layer, and the first electrodes and the second electrodes are obtained after volatilizing a solvent, and the conducting substance comprises a silver nanowire, a carbon nanotube or graphene.

5. The triboelectric nanogenerator based on a 4D printing technology according to claim 1, wherein longitudinal cross-sections of the first substrate layer and the second substrate layer are polygonal or curved edge-shaped.

6. The triboelectric nanogenerator based on a 4D printing technology according to claim 1, wherein a central angle corresponding to each of the friction units is a, and two adjacent friction units are spaced at a same central angle b; and a central angle corresponding to each of the first electrodes is c, and a central angle corresponding to each of the second electrodes is e, wherein a=c=d and b=c+2*e.

7. A self-powered sensing system, wherein the triboelectric nanogenerator based on a 4D printing technology according to claim 1 is assembled at a joint; the first triboelectric component and the second triboelectric component are assembled at one side of the joint; when the joint moves, the joint drives one of the triboelectric components and the other triboelectric component to rotate relative to each other, so as to generate an alternating current signal, and an angle of the joint movement can be deduced according to a characteristic of the alternating current signal.

8. A method for detecting a rotation angle of a joint of the self-powered sensing system according to claim 7, comprising the following steps:
S1: measuring phases of electric signals outputted by the triboelectric nanogenerator at different rotation angles, respectively, and establishing a correspondence table between the rotation angle and the phases of the outputted electric signals;
S2: acquiring the outputted electric signals of the self-powered sensing system mounted at the joint in real time, and performing smoothing and noise reduction processing on the outputted electric signals;
S3: detecting phase information corresponding to the outputted electric signals subjected to smoothing and noise reduction processing in S2;
S4: matching the phase information of the outputted electric signals according to the correspondence table between the rotation angle and the phases of the outputted electric signals calibrated in S1; and
S5: acquiring the rotation angle of the joint according to a matching result between the phase information of the outputted electric signals and the rotation angles in S4.

9. The method for detecting a rotation angle of a joint of the self-powered sensing system according to claim 8, wherein in S2, a plurality of self-powered sensing systems are mounted at the joint simultaneously, and correspondingly, a mean value of the phase information corresponding to the outputted electric signals subjected to smoothing and noise reduction processing is solved in S3 as final phase information.

10. A method for preparing the triboelectric nanogenerator based on a 4D printing technology according to claim 1, wherein comprising the following steps:
S1: designing an independent layer-based triboelectric nanogenerator model;
S2: performing force analysis on a working process of the model after modeling and performing a simulation test on distribution of an electric potential field;
S3: importing the tested model into slicing software for slicing and layering, selecting a processing sequence according to an actual structure of the model and generating a processing instruction;
S4: importing the processing instruction into a 3D printer to complete print processing of the first triboelectric component and the second substrate layer, respectively; if a printed product in a processing process does not meet a use requirement, returning to S1 to complete design and simulation test of the model again and generating a new processing instruction;
S5: spraying a volatile solution doped with a conducting substance to the surface of the second substrate layer by using a spraying machine after print processing, and volatilizing a solvent to obtain the first electrodes and the second electrodes; and
S6: assembling the first substrate layer and the second substrate layer with the first electrodes and the second electrodes into the triboelectric nanogenerator.

11. A self-powered sensing system, wherein the triboelectric nanogenerator based on a 4D printing technology according to claim 2 is assembled at a joint; the first triboelectric component and the second triboelectric component are assembled at one side of the joint; when the joint moves, the joint drives one of the triboelectric components and the other triboelectric component to rotate relative to each other, so as to generate an alternating current signal, and an angle of the joint movement can be deduced according to a characteristic of the alternating current signal.

12. A method for detecting a rotation angle of a joint of the self-powered sensing system according to claim 11, comprising the following steps:
S1: measuring phases of electric signals outputted by the triboelectric nanogenerator at different rotation angles, respectively, and establishing a correspondence table between the rotation angle and the phases of the outputted electric signals;
S2: acquiring the outputted electric signals of the self-powered sensing system mounted at the joint in real time, and performing smoothing and noise reduction processing on the outputted electric signals;
S3: detecting phase information corresponding to the outputted electric signals subjected to smoothing and noise reduction processing in S2;
S4: matching the phase information of the outputted electric signals according to the correspondence table between the rotation angle and the phases of the outputted electric signals calibrated in S1; and
S5: acquiring the rotation angle of the joint according to a matching result between the phase information of the outputted electric signals and the rotation angles in S4.

13. The method for detecting a rotation angle of a joint of the self-powered sensing system according to claim 12, wherein in S2, a plurality of self-powered sensing systems are mounted at the joint simultaneously, and correspondingly, a mean value of the phase information corresponding to the outputted electric signals subjected to smoothing and noise reduction processing is solved in S3 as final phase information.

14. A self-powered sensing system, wherein the triboelectric nanogenerator based on a 4D printing technology according to claim 3 is assembled at a joint; the first triboelectric component and the second triboelectric component are assembled at one side of the joint; when the joint moves, the joint drives one of the triboelectric components and the other triboelectric component to rotate relative to each other, so as to generate an alternating current signal, and an angle of the joint movement can be deduced according to a characteristic of the alternating current signal.

15. A method for detecting a rotation angle of a joint of the self-powered sensing system according to claim 14, comprising the following steps:
S1: measuring phases of electric signals outputted by the triboelectric nanogenerator at different rotation angles, respectively, and establishing a correspondence table between the rotation angle and the phases of the outputted electric signals;
S2: acquiring the outputted electric signals of the self-powered sensing system mounted at the joint in real time, and performing smoothing and noise reduction processing on the outputted electric signals;
S3: detecting phase information corresponding to the outputted electric signals subjected to smoothing and noise reduction processing in S2;
S4: matching the phase information of the outputted electric signals according to the correspondence table between the rotation angle and the phases of the outputted electric signals calibrated in S1; and
S5: acquiring the rotation angle of the joint according to a matching result between the phase information of the outputted electric signals and the rotation angles in S4.

16. The method for detecting a rotation angle of a joint of the self-powered sensing system according to claim 15, wherein in S2, a plurality of self-powered sensing systems are mounted at the joint simultaneously, and correspondingly, a mean value of the phase information corresponding to the outputted electric signals subjected to smoothing and noise reduction processing is solved in S3 as final phase information.

17. A self-powered sensing system, wherein the triboelectric nanogenerator based on a 4D printing technology according to claim 4 is assembled at a joint; the first triboelectric component and the second triboelectric component are assembled at one side of the joint; when the joint moves, the joint drives one of the triboelectric components and the other triboelectric component to rotate relative to each other, so as to generate an alternating current signal, and an angle of the joint movement can be deduced according to a characteristic of the alternating current signal.

18. A method for detecting a rotation angle of a joint of the self-powered sensing system according to claim 17, comprising the following steps:
S1: measuring phases of electric signals outputted by the triboelectric nanogenerator at different rotation angles, respectively, and establishing a correspondence table between the rotation angle and the phases of the outputted electric signals;
S2: acquiring the outputted electric signals of the self-powered sensing system mounted at the joint in real time, and performing smoothing and noise reduction processing on the outputted electric signals;
S3: detecting phase information corresponding to the outputted electric signals subjected to smoothing and noise reduction processing in S2;
S4: matching the phase information of the outputted electric signals according to the correspondence table between the rotation angle and the phases of the outputted electric signals calibrated in S1; and
S5: acquiring the rotation angle of the joint according to a matching result between the phase information of the outputted electric signals and the rotation angles in S4.

19. The method for detecting a rotation angle of a joint of the self-powered sensing system according to claim 18, wherein in S2, a plurality of self-powered sensing systems are mounted at the joint simultaneously, and correspondingly, a mean value of the phase information corresponding to the outputted electric signals subjected to smoothing and noise reduction processing is solved in S3 as final phase information.

20. A self-powered sensing system, wherein the triboelectric nanogenerator based on a 4D printing technology according to claim 5 is assembled at a joint; the first triboelectric component and the second triboelectric component are assembled at one side of the joint; when the joint moves, the joint drives one of the triboelectric components and the other triboelectric component to rotate relative to each other, so as to generate an alternating current signal, and an angle of the joint movement can be deduced according to a characteristic of the alternating current signal.
